(19) United States
(12) Patent Application Publication
(10) Pub. No.: US 2015/0112260 A1
(43) Pub. Date: Apr. 23, 2015

(54) THERMAL AND NEAR INFRARED DETECTION OF BLOOD VESSELS

(71) Applicant: ELBIT SYSTEMS LTD., Haifa (IL)

(72) Inventors: Ofer DAVID, Haifa (IL); Ron Schneider, Haifa (IL)

(21) Appl. No.: 14/507,865

(22) Filed: Oct. 7, 2014

Related U.S. Application Data
(60) Provisional application No. 61/891,906, filed on Oct. 17, 2013.

(51) Int. Cl. A61M 5/42; A61F 7/00; A61B 5/00

(52) U.S. Cl. CPC A61M 5/427; A61B 5/0064; A61F 7/007; A61F 2007/0087
USPC 604/116

(57) ABSTRACT

Systems and methods are provided for non-invasive detection of blood vessels. The systems and methods cool uniformly a tissue volume below a skin region for a specified cooling period and then image vessel thermal footprints of vessels below the skin as they heat up the skin region. The systems comprise a thermal imaging device configured to image the skin region after the cooling period, an image processor arranged to identify, in images captured by the thermal imaging device, which arise on the skin region after discontinuation of the cooling, and displaying means configured to present the identified vessel thermal footprints. The system and methods may analyze the spatio-temporal patterns of the natural heating of the skin surface to derive data on the location of the vessels under the skin. Three dimensional (3D) imaging optics and techniques may further enhance the vessel imaging.

### CROSS REFERENCE TO RELATED APPLICATIONS

This application claims the benefit of U.S. Provisional Patent Application No. 61/891,906 filed on Oct. 17, 2013, which is incorporated herein by reference in its entirety.

### BACKGROUND OF THE INVENTION

1. Technical Field

The present invention relates to the field of medical imaging, and more particularly, to location of blood vessels.

2. Discussion of Related Art

Locating blood vessels is necessary in various medical procedures—blood tests, intravenous therapy, and more. In many cases, it is difficult to locate blood vessels, for example in cases of elderly patients, dark-skinned patients, obese patients or drug abusers. The inability to detect blood vessels in such cases causes the caregiver to make extra attempts in needle insertion, which may cause among other problems—delay in IV treatment or blood tests diagnosis, discomfort to patients due to repetitive needle sticks, and higher cost due to increased personnel work-time and use of equipment.

### SUMMARY OF THE INVENTION

One aspect of the present invention provides a system comprising a cooling unit arranged to cool a tissue volume below a skin region for a specified cooling period, wherein the cooling of the skin region is uniform; a thermal imaging device configured to image the skin region after the cooling period; an image processor arranged to identify, in images captured by the thermal imaging device, vessel thermal footprints which arise on the skin region after discontinuation of the cooling, and displaying means configured to present the identified vessel thermal footprints. The processor's identification of the vessel thermal footprints may be based on identifying a spatial signature of the vessel in the image and/or on utilizing temporal changes in the detected footprint.

These, additional, and/or other aspects and/or advantages of the present invention are set forth in the detailed description which follows; possibly inferable from the detailed description; and/or learnable by practice of the present invention.

### BRIEF DESCRIPTION OF THE DRAWINGS

For a better understanding of embodiments of the invention and to show how the same may be carried into effect, reference will now be made, purely by way of example, to the accompanying drawings in which like numerals designate corresponding elements or sections throughout.

In the accompanying drawings:

FIGS. 1A and 1B are high level schematic illustrations of a system, according to some embodiments of the invention.

FIGS. 2A and 2B are high level schematic illustrations of three dimensional imaging system, according to some embodiments of the invention.

FIGS. 3A-3C, 4A and 4B are high level schematic illustrations of system with an opto-mechanical adaptor, according to some embodiments of the invention.

FIG. 5 is a high level schematic block diagram of system, according to some embodiments of the invention.

FIGS. 6A and 6B are schematically presented experimental results that indicate the operability and efficiency of system, according to some embodiments of the invention.

FIGS. 7A and 7B are illustrative images of the operability and efficiency of system, according to some embodiments of the invention.

FIG. 8 is a high level flowchart illustrating a method, according to some embodiments of the invention.

FIG. 9 is a high level flowchart illustrating a method, according to some embodiments of the invention.

### DETAILED DESCRIPTION OF THE INVENTION

Prior to the detailed description being set forth, it may be helpful to set forth definitions of certain terms that will be used hereinafter.

The term “tissue volume” as used in this application refers to a volume of the body which may include any type of tissue, as well as blood vessels and fluids in the vessels. Tissue volumes may have a skin boundary for non-invasive applications, or may be an internal tissue volume in invasive, e.g., surgical applications.

With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

Before at least one embodiment of the invention is explained in detail, it is to be understood that the invention is not limited in its application to the details of construction and the arrangement of the components set forth in the following description or illustrated in the drawings. The invention is applicable to other embodiments or of being practiced or carried out in various ways. Also, it is to be understood that the phraseology and terminology employed herein is for the purpose of description and should not be regarded as limiting.

Systems and methods are provided for non-invasive detection of blood vessels, according to certain embodiments. The systems and methods cool uniformly a tissue volume below a skin region for a specified cooling period and then capture vessel thermal footprints of vessels below the skin as they heat up the skin region naturally, after the cooling ceases. The systems comprise a thermal imaging device configured to image the skin region after the cooling period, an image processor arranged to identify, in images captured by the thermal imaging device, which arise on the skin region after discontinuation of the cooling, and displaying means configured to present the identified vessel thermal footprints. The system and methods may analyze the spatio-temporal patterns of heating the skin surface to derive data on the location of the vessels under the skin. Three dimensional (3D) imaging optics and techniques may further enhance the vessel imaging. Near infra-red (NIR) or visual range illumination may be directed via manipulators such as needles to enable monitoring spatial relations between the vessels and the inserted manipulators. The capturing of the NIR or visual image may be carried out using an imager sensitive in the visual or NIR spectral band (like a CCD (charge-coupled device), a CMOS (complementary metal-oxide-semiconductor device) etc.), which may be the same as the imager of the thermal imaging device or different therefrom.

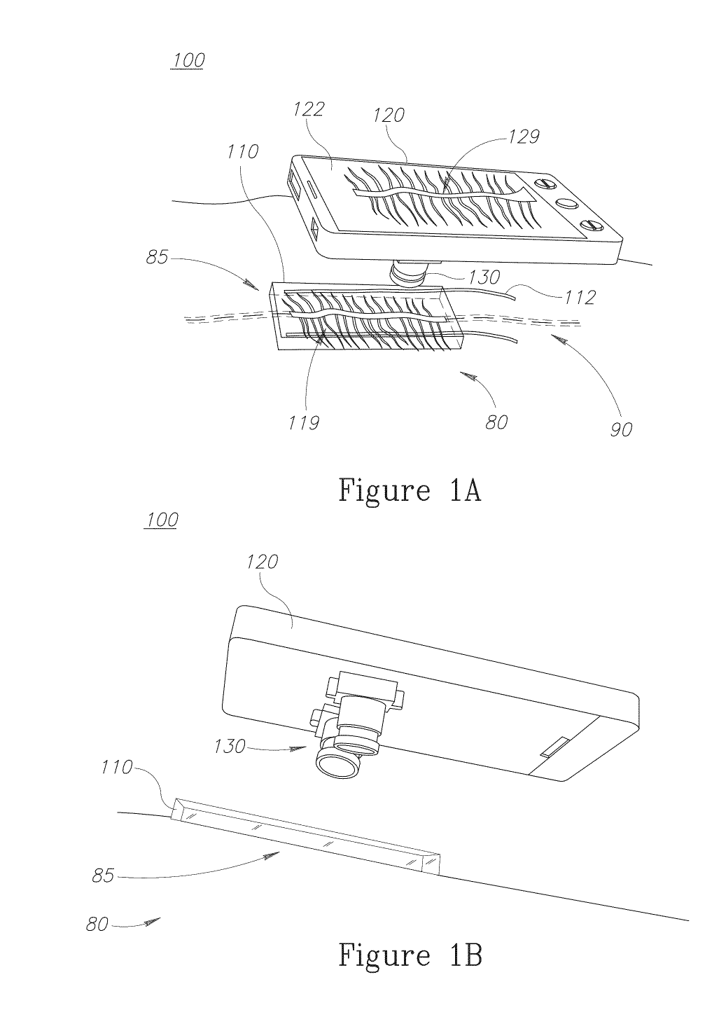

FIGS. 1A and 1B are high level schematic illustrations of a system 100 according to some embodiments of the invention. System 100 comprises a cooling unit 110 arranged to cool a tissue volume 80 below a skin region 85 for a specified cooling period. Cooling skin region 85 is carried out uniformly, with minimal or no temperature differences arising between parts of region 85. In certain embodiments, system 100 (and method 200 described below) may be applied to tissue volumes 80 which are not bound by skin but are internal tissue volumes. System 100 and method 200 may be applied to such tissues in a similar way, and the term “skin region” 85 is then understood as a part of the surface of the tissue volume. For example, system 100 and method 200 may be used in internal surgical procedures, e.g., laparoscopic surgery, automated, semi-automated or manual, and identify blood vessels below the surface to prevent damage or to operate the vessels controllably.

System 100 further comprises a thermal imaging device 120, which may comprise one or more cameras, configured to image skin region 85 after the cooling period to record the gradual differential heating up of skin region 85. In certain embodiments, thermal imaging device 120 comprises a LWIR (long wavelength infrared, e.g., operating in a range within 3-20 micrometers) thermal imaging device.

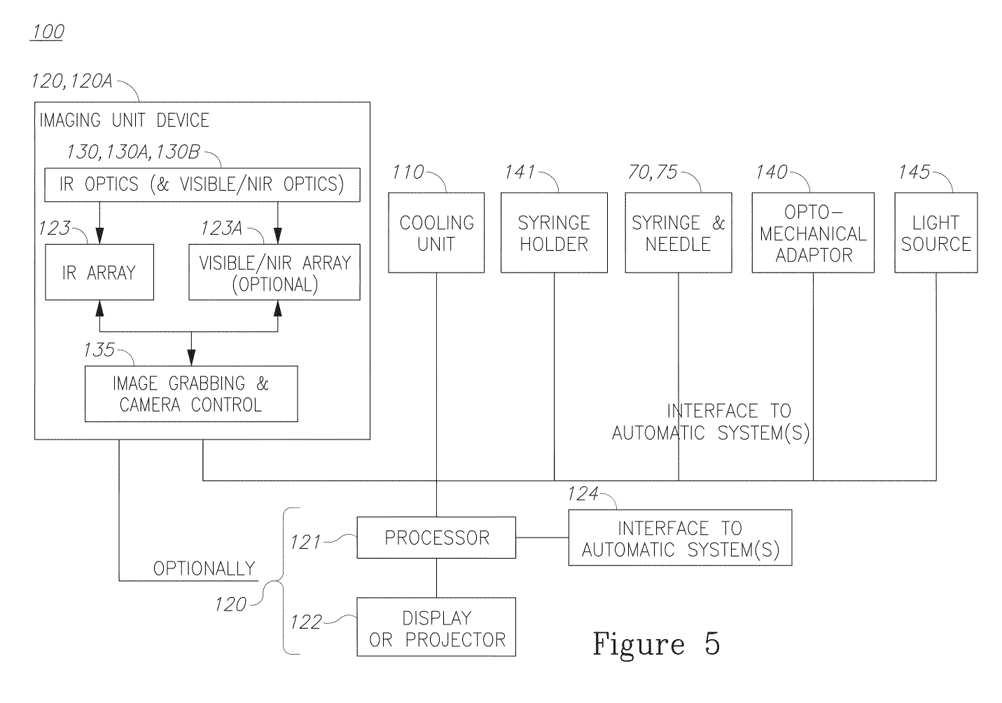

System 100 further comprises an image processor 121 (shown in FIG. 5) arranged to identify (129) in images captured by thermal imaging device 120, vessel thermal footprints 119 which arise on skin region 85 after discontinuation of the cooling. Identified vessel thermal footprints 129 may be used as indication of the location of vessels 90 within tissue volume 80 below the skin, which are otherwise not visible. Processor 121's identification of vessel thermal footprints 129 may be based on identifying a spatial signature of vessel 90 in the image and/or on utilizing temporal changes in detected footprint 129.

The cooling (or in some embodiments, heating) of the surface of the skin is configured to be uniform in the sense that no excessive surface temperature differences are formed which mask vessel thermal footprints 119 and prevent their detection 129. For example, the term “uniform” may be understood as indicating cooling (or heating) characterized by a small variability over the cooled surface, e.g., variability smaller than 0.1° C., or even smaller than 0.01° C. in certain embodiments. In certain embodiments however, the term “uniform” may be used to indicate variability smaller than 0.5° C., especially over large areas. In certain embodiments, a specific part of skin region 85 may be cooled or heated differently than the rest of skin region 85. Such part(s) may be used for calibration purposes. In certain embodiments, different parts of skin region 85 may be cooled (or heated) to different temperatures, in a controlled and uniform manner. For example, cooling temperature may be adapted to expected vessel depth in the respective part, or different temperatures may be applied for exploratory purposes. In either part, cooling may be uniform in the explained sense to prevent masking of vessel thermal footprints 119 by thermal surface noise.

System 100 further comprises displaying means 122 configured to present identified vessel thermal footprints 119, e.g., on a display of imaging device 120 or by projection on skin region 85. Displaying means 122 may comprise a monitor (e.g., a liquid crystal display, LCD, an organic light emitting diodes OLED display), a head mounted display (a small display optically in front of the user's eyes, e.g., using a digital light processing DLP projector) or any other form of display. The display may be two dimensional or possibly three dimensional (based on any method, such as differing colors or polarizations, directional pixels, etc., and see below). Displaying means 122 may be part of imaging device 120 or connected thereto with cables or wirelessly. The displayed images 120 of vessel thermal footprints 119 may be used to apply direct treatment such as injections, intravenous therapy or blood extraction procedures and to carry out diagnostics procedures.

In certain embodiments, system 100 may comprise a heating unit in place or in addition to cooling unit 110. The heating unit may uniformly heat skin region 85 and imaging device 120 and processor 121 may be configured to identify vessel thermal footprints 119 which arise on skin region 85 after discontinuation of the heating (as cooler regions). Certain embodiments comprise heating instead of cooling and applying respective processing.

The cooling may be carried out via direct contact with skin region 82 or indirectly, e.g. by cool air convection over skin region 85. For example, cooling unit 110 may employ direct contact cooling and then removed prior to the imaging. For example, cooling unit 110 may be cooled using gas, may comprise one or more thermal electric cooler(s) or employ any other method. Cooling unit 110 may have a disposable sterile part coming in touch with the skin or may be completely disposable. Cooling unit 110 may be part of or attached to a back side of imaging device 120. In case cooling unit 110 is gas operated, imaging device 120 may provide gas-filling possibilities.

In certain embodiments, cooling unit 110 may be transparent in the thermal imaging bandwidth (e.g., LWIR) and the imaging may be carried out therethrough. Cooling unit 110 may be transparent in the operation thermal band of imaging device 120 (e.g., any range within 3-14 micrometer). Non-limiting examples for materials having such transparency features are germanium, zinc selenide, zinc sulfide, sapphire, chalcogenide glass etc. Cooling unit 110 may be configured to cool skin region 85 uniformly or in a specified non-uniform pattern, and control the temperature of the skin during the imaging, which may accompany long duration injection procedures. Cooling unit 110 may comprise an opening (not shown) for a syringe to reach the skin therethrough. An additional sterile sheet (not shown) may be used between the opening and the skin. In certain embodiments, the transparent material may be plastic which may be made transparent in a respective range in the infrared.

For example, cooling unit 110 may comprise a cold package, e.g., holding cold material such as a fluid or a gel. In another example, cooling unit 110 may be thermoelectrically cooled (applying thermoelectric cooling TEC via wires 112). Cooling unit 110 may be part of imaging device 120 or may be connected thereto via cables or wirelessly.

In certain embodiments, cooling unit 110 may be configured to sterilize skin region 85.

In certain embodiments, cooling unit 110 may comprise multiple cooling subunits, each possibly configured to cool skin region 85 to a different temperature.

In certain embodiments, a tourniquet (not shown) may be used to enhance blood vessels 90 before cooling, during cooling or after the cooling, as the skin warms up.

In certain embodiments, cooling unit 110 may be configured to be employed without contact to skin region 85. For example, cooling unit 110 may cool by air convection, using cold gas flow to the skin. The cooling gas may be selected not to obscure the image in continuous applications, or the cooling gas may be quickly removed after the cooling phase in intermittent applications.

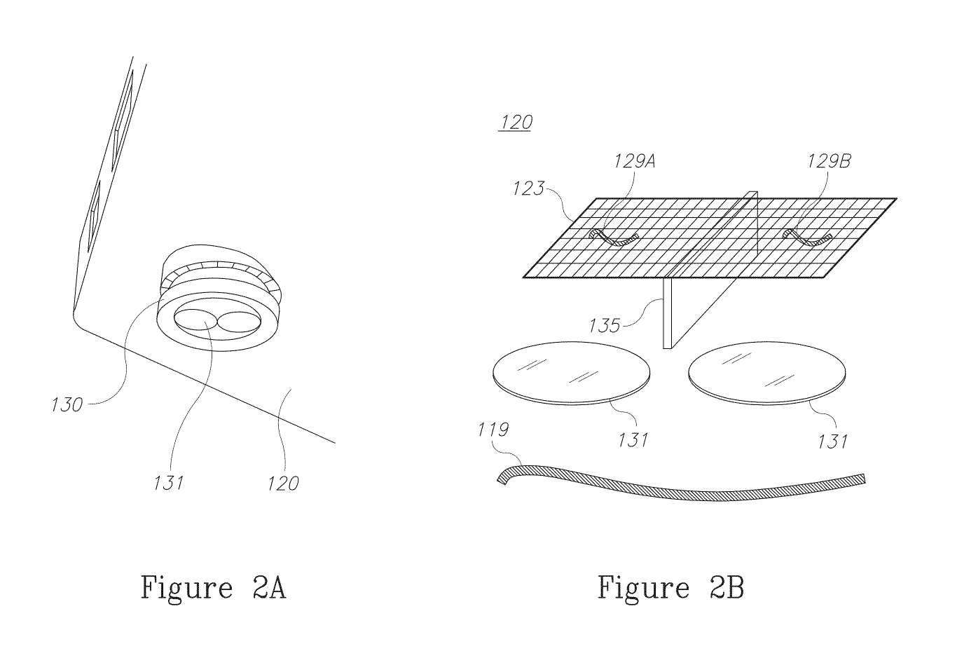

FIGS. 2A and 2B are high level schematic illustrations of three dimensional imaging system 100 according to some embodiments of the invention. Thermal imaging device 120 and image processor 121 may be arranged to produce three dimensional images of vessel thermal footprints 119. For example, thermal imaging device 120 may comprise optics 130 with two or more spatially separate imaging elements 131 such as different apertures, different lenses or different cameras. FIG. 2A schematically illustrates embodiments with two lenses or two cameras 131 and FIG. 2B schematically illustrates embodiments with a split field of view (elements 131 may be different lenses or different apertures). Two images 129A, 129B of vessel thermal footprints 119 may be captured in different regions of sensor 123 and processed to yield a three dimensional image (a stereo image may be produced using the parallax between elements 131). Possibly a physical barrier 135 may separate the fields of view or the regions on sensor 123 and prevent stray or ghost light from passing between the two sides of sensor 123.

In certain embodiments, thermal imaging device 120 and sensor 123 may be uncooled.

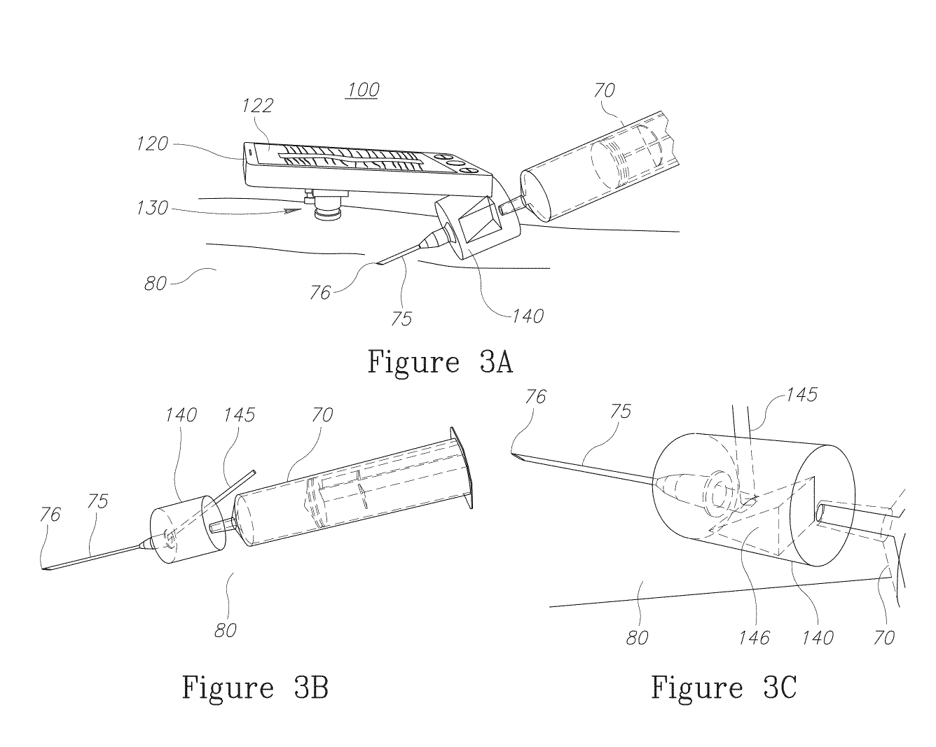

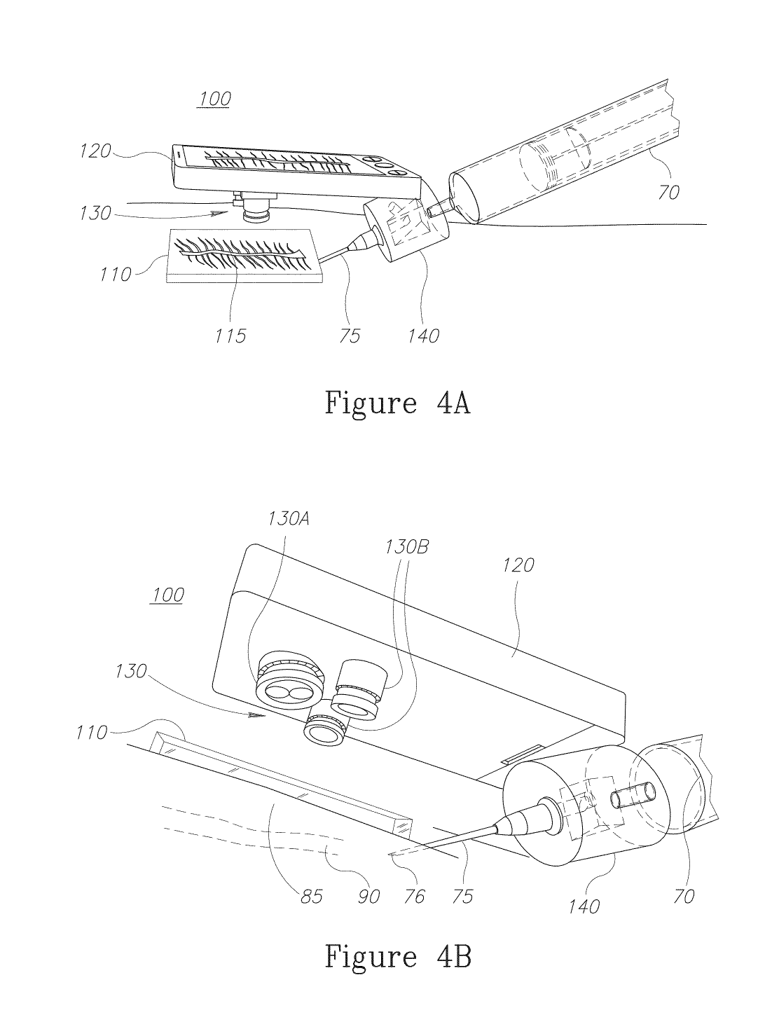

FIGS. 3A-3C, 4A and 4B are high level schematic illustrations of system 100 with an opto-mechanical adaptor 140, according to some embodiments of the invention. Opto-mechanical adaptor 140 is interconnected between a syringe 70 and a needle 75 and is configured to maintain fluid communication between syringe 70 and needle 75 therethrough, and to direct illumination 145 into needle 75 (e.g., when needle 75 is filled with air). Illumination 145 may be produced externally or internally (within adaptor 140), e.g., by laser(s), light emitting diode(s) LED(s) or incandescent sources. System 100 may further comprise a second, NIR (near infrared) or visual range imaging device 120A, possibly implemented within thermal imaging device 120, which is configured to detect the directed illumination or reflections thereof. Second imaging device 120A may comprise filters appropriate to let through only relevant parts of illumination 145 and its reflections, e.g., the filter may enable imaging of reflected light only. Second imaging device 120A may comprise one or more imaging elements, e.g., to capture illumination 145 and its reflections three dimensionally.

For example, illumination 145 emanating from a tip 76 of needle 75 may be imaged, or reflections and transmissions of illumination 145 off inner tissues or vessels may be imaged. Image processor 121 may be configured to fuse images from thermal and second imaging devices 120, 120A. Second imaging device 120A may augment the thermal imaging by tissue depth data achieved from internal illumination to reach higher contrast, three dimensional data and three dimensional relations between bodily and external elements. Optics 130 of either or both imaging devices 120, 120A may comprise a single lens for 2D imaging, two lenses for 3D imaging or a special lens with two apertures 131 for 3D imaging. One, two or more sensors, same or different, may be applied to any of imaging devices 120, 120A. For example, FIG. 4B schematically illustrates in a non-limiting manner, configurations with thermal imaging device 120 optics 130A having two optical elements 131 and second imaging device 120A integrated within thermal imaging device 120 and having two imaging devices (cameras) 130B. Any other combination of the possibilities described above is likewise part of the present disclosure.

In certain embodiments, system 100 may comprise opto-mechanical adaptor 140 and second, NIR (near infrared) or visual range imaging device 120A (possibly with associated image processor 121 and displaying means 122), without cooling unit 110 and thermal imaging device 120.

In certain embodiments, system 100 may comprise a syringe holder 141 (shown schematically in FIG. 5 below) that holds and stabilizes syringe 70 and/or adapter 140. Holder 141 may be mechanically associated with any part of system 100, e.g., with imaging devices 120, 120A, cooling unit 110, displaying means 122 or any external mechanism. Syringe 70 may be affixed to any element of system 100, e.g., to imaging devices 120.

Opto-mechanical adaptor 140 and illumination 145 may be configured to yield accurate illumination and imaging of vessel 90 once syringe 70 is inserted into the skin. Illumination 145 through needle 75 overcomes the strong reflection from the skin top surface which dazzles or saturates other devices. In certain embodiments, illumination 145 may comprise more than one wavelength or wavelength ranges to enable separation of types of objects in tissue region 80.

In certain embodiments, illumination 145 emanating from the tip of needle 75 may be easily detected and used to position needle tip 76 with respect to vessel 90. As needle 75 penetrated region 85, ever deeper vessels may be illuminated and captured to enhance the processed image of the region.

Opto-mechanical adaptor 140 may be designed as an add-on mountable onto a standard syringe 70. Adaptor 140 may be a disposable plastic optical interface, focusing illumination 145 from a light source into the needle cavity. Adaptor 140 may be part of syringe 70 and/or needle 75.

In certain embodiments, the source of illumination 145 and second imaging device 120A may be temporally synchronized to increase signal to noise ratio (avoid background noise) and improve the detection reliability. Synchronization may achieved, for example, (i) by pulsing illumination 145 into the camera integration time between camera frames, (ii) by alternating illumination pulses and frame capturing multiple times during the capturing of each frame, with camera integration time overlapping the light source pulses, (iii) by applying very narrow illumination pulses which are synchronized with narrow camera integration times or (iv) by closing the camera shutter during illumination pulses 145. A second source of illumination may be used to further enhance the captured images.

Illumination 145 may be used to detect puncturing of vessel 90, as needle tip detection may be blocked by blood flowing into (or out of) needle 75. Image processor 121 and/or system 100 may be configured to detect changes of needle tip illumination to indicate vessel puncturing. Adaptor 140 may be designed to enable free flow between syringe 70 and needle 75 without obstruction and without contamination risk.

Illumination may be directed, e.g., in an oblique path indicated schematically in FIG. 3B and/or using optical elements 146 such as mirrors, beam splitters or lenses (e.g., to focus illumination 145 at the needle tip).

FIG. 5 is a high level schematic block diagram of system 100, according to some embodiments of the invention. The schematic block diagram schematically illustrates the presented components of system 100, including imaging device 120 which may include both thermal imaging device 120 and second imaging device 120A embodied in one device or associated with each other. Imaging device 120 may comprise respective optics 130 in any of the embodiments (two or three dimensional, integrated or associated with each other, optical elements such as filters, beam splitters etc.), respective detectors or sensors 123, 123A for the relevant wavelength ranges (e.g., ranges within 3-20 μm for thermal imaging device 120, 0.7-1.1 μm (or shorter wavelengths) for visual/NIR devices 120A) and control modules 135 for controlling image grabbing parameters and camera operating parameters such as gain and exposure time. Processor 121 and/or display 122 may be part of imaging device 120 or associated therewith. Images taken in different wavelength ranges may be used to enhance each other (e.g., by providing additional measurements or references) and/or combined into enhanced fused. System 100 may further comprise an interface to automatic system(s) 124 such as robotic operating machines and be configured to automatically detect and/or alert concerning blood vessels, provide information to such automatic system(s) and/or be operated by such systems according to a respective operational procedure. Cooling unit 110 is also associated with imaging device, either as part thereof or provided in a kit. Possibly several cooling units and/or several cooling unit elements may be included in such kit, for example disposable gas containers for gas cooled cooling units 110, electric components for TEC etc. Opto-mechanical adaptor 140 and possibly light source 145, syringe holder 141 and/or syringe and needle 70, 75 may also be provided in such kit, in a medically usable form (sterilized, disposable, etc.). The modules of system 100 may be arranged to enhance the comfort and efficiency of use. For example, modules may be mechanically fixated (e.g., rigidly connected) to reduce the handling load on the physician, modules may be incorporated in or as a head mounted device, and/or be associated with auxiliary mechanical elements such as fixtures or arms. In certain embodiments, cooling unit 110 may be completely separated from the rest of the modules.

System 100 may be adapted for use by robotic manipulation systems and be fully automated, integrating image processing results into the software and algorithms operated by the robotic system.

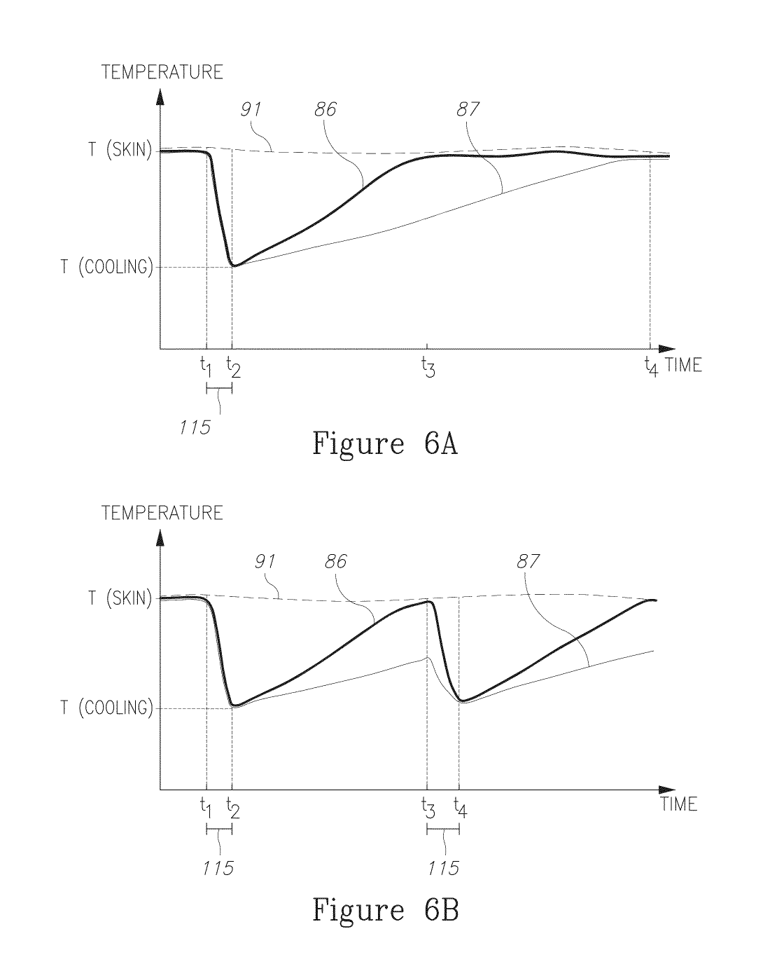

FIGS. 6A and 6B are schematically presented experimental results that indicate the operability and efficiency of system 100, according to some embodiments of the invention. Curve 91 schematically represents the temperature of actual blood vessel 90, which remains pretty constant during the procedure due to the continuous blood supply at a stable temperature (e.g., body temperature—the blood vessel heat gradient is smaller than the surrounding heat gradient due to the cooling, since blood vessels tend to change their temperature in a relatively slower rate than the surrounding tissue). Curves 86, 87 schematically represent the temperature of the skin in skin region 85: Curve 86 describes the temperature of the skin portion above blood vessel 90 and curve 87 describes the temperature of the skin portion which is not above blood vessel 90 (i.e., farther removed therefrom with respect to the skin portion above blood vessel 90). Cooling takes place during cooling period 115 (t₁ to t₂) as result of the application and activation of cooling unit 110. For example, cooling may reduce skin temperature in 3-5° C. and be carried out for periods 115 between 10 and 120 seconds. Cooling is uniform for all contacted skin locations. After the end of cooling period 115, either due to the removal of cooling unit 110 or due to the deactivation thereof, skin temperature rises at different paces in different locations of skin region. The skin temperature above vessel 90 rises relatively steeply as illustrated by curve 86, while the skin temperature not above vessel 90 rises slower as illustrated by curve 87. During the natural heating up of skin region 85, a temperature difference is created between skin areas above vessel 90 and other skin areas (see e.g., at time t₃ in FIG. 6A), yielding detectable thermal signals that represent vessel thermal footprints 119. The larger the temperature difference between curves 86 and 87 over time, the better are the detection capabilities and the efficiency of data processing. Certain embodiments enhance these differences by configuring the cooling accordingly. Finally (at time t₄), skin temperature over whole skin region 85 equalizes and reaches its former value, e.g., similar to blood vessel temperature 91.

FIG. 6B schematically represents experimental results for reiterated cooling as periods 115 t₁-t₂, t₃-t₄ which result in recurring temperature differences that can be monitored over time to enhance vessel identification. In certain embodiments, the cooling temperature may be configured according to imaging results, e.g., cooling may be increased when imaging is of reduced quality. Processor 121 may adapt manually or automatically the cooling temperature according to imaging performance, providing feedback control of the operation of system 100. In certain embodiments, cooling may be continuous (long period 115) and/or fluctuate in intensity (the cooling temperature may vary during the procedure. In this case too, imaging quality may be used to adapt the cooling parameters and characteristics to optimize image quality and vessel detection efficiency.

Temperature difference in the cooling behavior of skin regions, which depends of the location of the respective skin region with respect to blood vessels, is observed upon heating skin region (similarly to the case described above of cooling the skin region) as well. Furthermore, such temperature differences which are indicative to locations of blood vessels below the surface are also detectable upon applying system 100 to internal tissue volumes 80, where tissue surface is used for images in place of skin region 85. In certain embodiments, heating is applied in place of cooling and image processing is configured accordingly. In certain embodiments, system 100 may be used at internal tissue volumes with respect to their surfaces in place of the skin region.

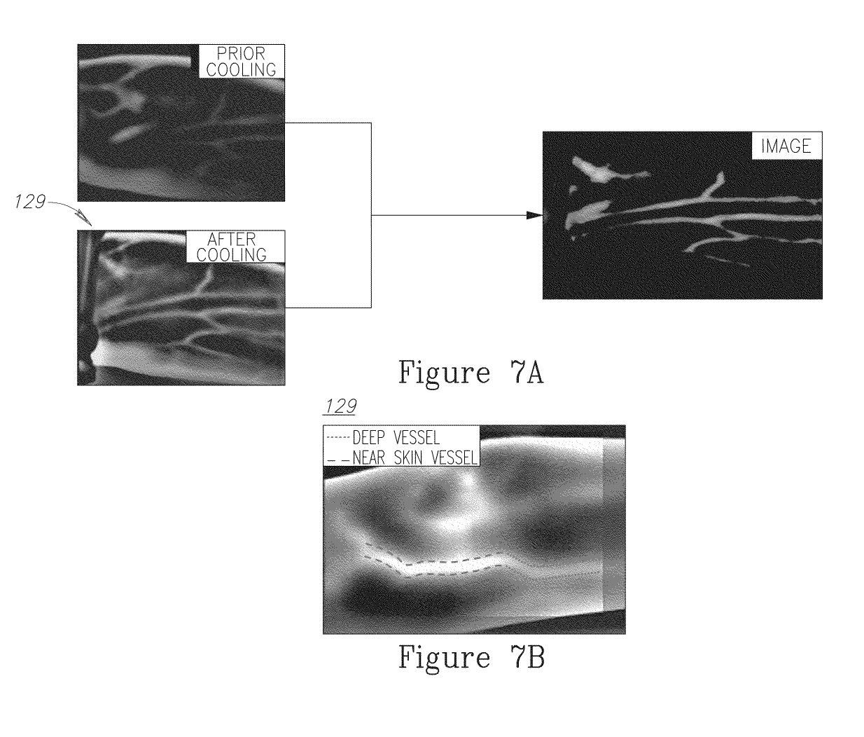

FIGS. 7A and 7B are illustrative images of the operability and efficiency of system 100, according to some embodiments of the invention. FIGS. 7A and 7B are illustrative thermal images of skin region 85 before cooling (top left in FIG. 7A), after cooling with imaged thermal vessel footprints 129 (bottom left in FIG. 7A), and a processed image of the vessels (right side of FIG. 7A). The processed image may be enhanced using past images and the vessels may be extracted and enhanced e.g., by tracking the differences between sequential images and detecting the evolving differences (see FIGS. 6A, 6B for the development of the temperature differences between locations on skin surface 85). The thermal imaging may take into consideration the ambient temperature and/or the operation of system 100 may be carried out at regulated temperatures to provide optimal conditions for the thermal imaging.

FIG. 7B schematically illustrates imaged thermal vessel footprints 129 at higher thermal resolution which provide some depth information for vessels 90. Depth information may be derived from the blood vessel image sharpness (or blur), being a function of the vessel depth. In addition, the temporal change of vessel sharpness after cooling depends on its depth and may be estimated from sequential images. For example, the inventors have found out that the rate of vessel apparent width change may be used to indicate their depth, as the vessel thermal footprints of vessels which are closer to the surface increase in width faster than do thermal footprints of deeper vessels. Depth information may be enhanced by applying 3D imaging (e.g., with two or more imaging elements) or by detecting illumination directed through needle 75.

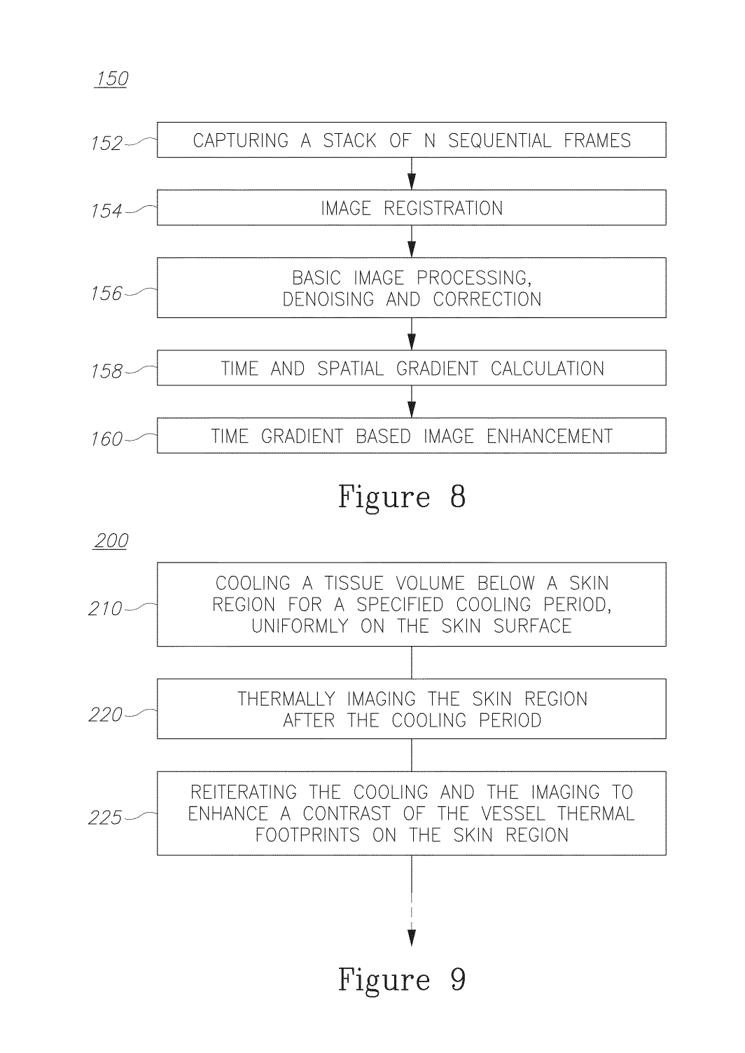

FIG. 8 is a high level flowchart illustrating a method 150, according to some embodiments of the invention. Method 150 may be partially or fully implemented in hardware. Certain embodiments comprise a non-transitory computer readable storage medium having computer readable program embodied therewith which is configured to carry out at least parts of method 150.

Method 150 comprises capturing stacks of sequential frames (stage 152), registrating the images (stage 154), e.g., to compensate for camera or object movements, processing the images to remove noise and correct image features (stage 156) and then carrying out tempo-spatial gradient calculation (stage 158) that lead to respective image enhancements (stage 160) as explained above, providing data concerning vessel characteristics. In certain embodiments, the time gradient of each pixel may be calculated 158 by comparison between frames, and using each pixel's time gradient data, pixels suspected as “blood-vessel pixels” may be identified by their low gradients. A local contrast enhancement may be performed in the relevant areas, to make the blood vessels more apparent. The image processing methods may include standard techniques of image integration, contrast enhancement, shape recognition, threshold and fusion. Moreover, the original image may be enhanced 160 by measuring the heat time-gradient distribution between frames. The processing may be based on correlation between frames.

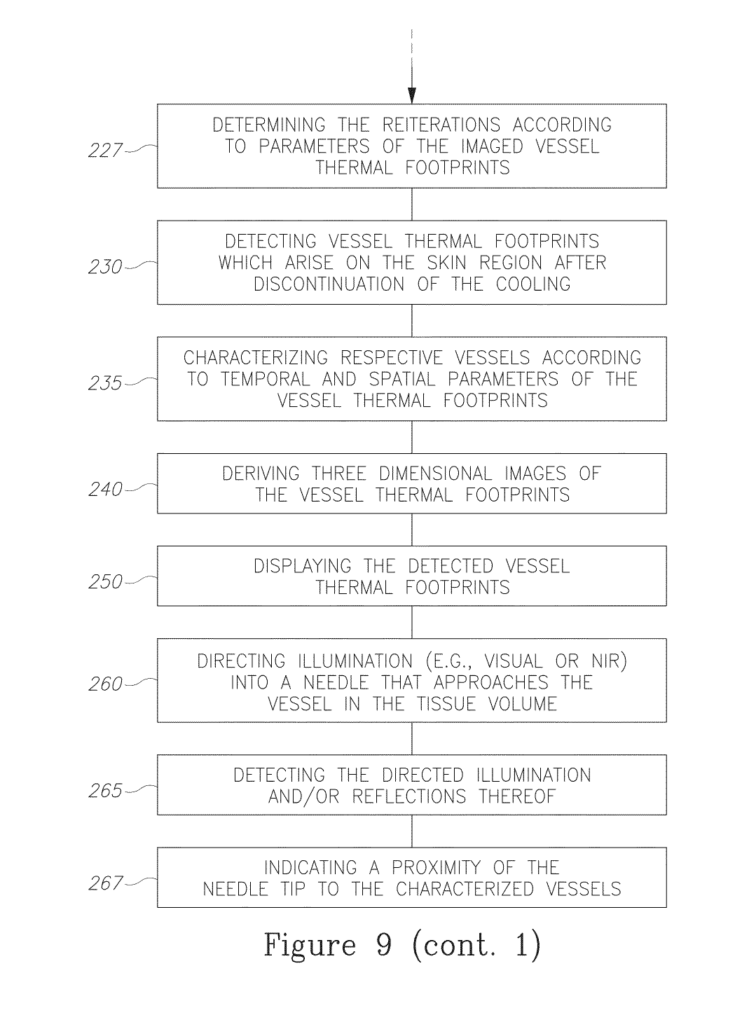

FIG. 9 is a high level flowchart illustrating a method 200, according to some embodiments of the invention. Method 200 may be partially or fully implemented in hardware. Certain embodiments comprise a non-transitory computer readable storage medium having computer readable program embodied therewith which is configured to carry out at least parts of method 200. Method 200 comprises any of the following stages: cooling a tissue volume below a skin region for a specified cooling period, wherein the cooling of the skin region is uniform (stage 210), imaging the skin region in infrared (e.g., LWIR) after the cooling period (stage 220) to detect vessel thermal footprints which arise on the skin region after discontinuation of the cooling (stage 230), and displaying the detected vessel thermal footprints (stage 250). Method 200 may comprise some or all of the stages of method 150.

Method 200 may further comprise reiterating cooling 210 and imaging 220 to enhance a contrast of the vessel thermal footprints in the skin region (stage 225). Method 200 may comprise determining reiterations 225 according to parameters of the imaged vessel thermal footprints (stage 227).

Method 200 may further comprise characterizing respective vessels according to temporal and spatial parameters of the vessel thermal footprints (stage 235).

Method 200 may further comprise deriving three dimensional images of the vessel thermal footprints (stage 240).

In certain embodiments, method 200 may further comprise directing illumination into a needle directed at a vessel in the tissue volume (stage 260) and detecting the directed illumination or reflections thereof (stage 265). In certain embodiments, method 200 may comprise characterizing respective vessels according to temporal and spatial parameters of the vessel thermal footprints (stage 235), and indicating a proximity of a tip of the needle to the characterized vessels (stage 267).

Data processing stages and control stages may be implemented by respective processors and algorithms may be implemented by respective computer program product(s) comprising a computer usable medium having computer usable program code tangibly embodied thereon, the computer usable program code configured to carry out at least part of the respective stages.

In the above description, an embodiment is an example or implementation of the invention. The various appearances of “one embodiment”, “an embodiment”, “certain embodiments” or “some embodiments” do not necessarily all refer to the same embodiments.

Although various features of the invention may be described in the context of a single embodiment, the features may also be provided separately or in any suitable combination. Conversely, although the invention may be described herein in the context of separate embodiments for clarity, the invention may also be implemented in a single embodiment.

Certain embodiments of the invention may include features from different embodiments disclosed above, and certain embodiments may incorporate elements from other embodiments disclosed above. The disclosure of elements of the invention in the context of a specific embodiment is not to be taken as limiting their used in the specific embodiment alone.

Furthermore, it is to be understood that the invention can be carried out or practiced in various ways and that the invention can be implemented in certain embodiments other than the ones outlined in the description above.

The invention is not limited to those diagrams or to the corresponding descriptions. For example, flow need not move through each illustrated box or state, or in exactly the same order as illustrated and described.

Meanings of technical and scientific terms used herein are to be commonly understood as by one of ordinary skill in the art to which the invention belongs, unless otherwise defined.

While the invention has been described with respect to a limited number of embodiments, these should not be construed as limitations on the scope of the invention, but rather as exemplifications of some of the preferred embodiments. Other possible variations, modifications, and applications are also within the scope of the invention. Accordingly, the scope of the invention should not be limited by what has thus far been described, but by the appended claims and their legal equivalents.

1. A system comprising:
a cooling unit arranged to cool a tissue volume below a skin region for a specified cooling period, wherein the cooling of the skin region is uniform,
a thermal imaging device configured to image the skin region after the cooling period, and
an image processor arranged to identify, in images captured by the thermal imaging device, vessel thermal footprints which arise on the skin region after discontinuation of the cooling.

2. The system of claim 1, wherein the image processor is further arranged to characterize respective vessels according to temporal and spatial parameters of the vessel thermal footprints.

3. The system of claim 1, further comprising a displaying means configured to present the identified vessel thermal footprints.

4. The system of claim 1, wherein a spectral sensitivity of the thermal imaging device is in LWIR (long wavelength infrared).

5. The system of claim 1, wherein the cooling unit employs direct contact cooling and is removed prior to the imaging.

6. The system of claim 5, wherein the cooling unit is a thermo electric cooler.

7. The system of claim 1, wherein the cooling unit is transparent in an imaging infrared range and the imaging is carried out therethrough.

8. The system of claim 1, wherein the cooling unit is employed without contact to the skin region.

9. The system of claim 1, wherein the thermal imaging device and the image processor are arranged to produce three dimensional images of the vessel thermal footprints.

10. The system of claim 9, wherein the thermal imaging device comprises two or more spatially separate imaging elements.

11. The system of claim 1, further comprising:
an opto-mechanical adaptor, interconnected between a syringe and a needle and configured to direct illumination into the needle, and
a second, NIR (near infrared) or visual spectral band imaging device configured to detect the directed illumination or reflections thereof.

12. The system of claim 11, wherein the second imaging device is CCD or CMOS based.

13. The system of claim 11, wherein the image processor is configured to fuse images from the thermal and the second imaging devices.

14. The system of claim 1, wherein the displaying means are projection on the skin region.

15. A system comprising:
an opto-mechanical adaptor, interconnected between a syringe and a needle and configured to direct illumination into the needle, and
a NIR (near infrared) or visual spectral band imaging device configured to detect the directed illumination or reflections thereof.

16. The system of claim 15, wherein the imaging device is CCD or CMOS based.

17. A method comprising:
cooling a tissue volume below a skin region for a specified cooling period, wherein the cooling of the skin region is uniform, and
imaging the skin region in infrared after the cooling period to detect vessel thermal footprints which arise on the skin region after discontinuation of the cooling.

18. The method of claim 17, further comprising displaying the detected vessel thermal footprints.

19. The method of claim 17, further comprising reiterating the cooling and the imaging to enhance a contrast of the vessel thermal footprints in the skin region.

20. The method of claim 19, further comprising determining the reiterations according to parameters of the imaged vessel thermal footprints.

21. The method of claim 17, further comprising characterizing respective vessels according to temporal and spatial parameters of the vessel thermal footprints.

22. The method of claim 17, further comprising deriving three dimensional images of the vessel thermal footprints.

23. The method of claim 17, further comprising directing illumination into a needle directed at a vessel in the tissue volume and detecting the directed illumination or reflections thereof.

24. The method of claim 23, further comprising characterizing respective vessels according to temporal and spatial parameters of the vessel thermal footprints, and indicating a proximity of a tip of the needle to the characterized vessels.

25. The method of claim 23, wherein the directed illumination is in near infrared or visual range.
